(19) United States
(12) Patent Application Publication
(10) Pub. No.: US 2025/0002861 A1
(43) Pub. Date: Jan. 2, 2025

(54) IMPROVED ADIPOSE STEM CELL-DERIVED SECRETOME THERAPY FOR AMYOTROPHIC LATERAL SCLEROSIS AND OTHER NEURDEGENERATIVE DISORDERS

(71) Applicants: The Trustees of Indiana University, Bloomington, IN (US); U.S. GOVERNMENT, THE DEPT. OF VETERANS AFFAIRS, Washington, DC (US)

(72) Inventor: Chandler Walker, Indianapolis, IN (US)

(21) Appl. No.: 18/709,235

(22) PCT Filed: Nov. 3, 2022
(86) PCT No.: PCT/US2022/048806

§ 371 Date: May 10, 2024

Related U.S. Application Data
(60) Provisional application No. 63/278,744, filed on Nov. 12, 2021.

(51) Int. Cl. C12N 5/0775; A61K 35/28; A61P 25/28

(52) U.S. Cl. CPC C12N 5/0667; A61K 35/28; A61P 25/28

(57) ABSTRACT

The general field of the present disclosure are novel approaches to producing a cell-based therapy wherein certain secreted proteins and extracellular components are isolated from adipose-derived stem cells. Specifically, the present disclosure provides novel therapeutics to target and treat neurodegenerative diseases such as amyotrophic lateral sclerosis.

### REFERENCE TO GOVERNMENT GRANTS

This invention was made with government support from the Department of Veterans Affairs of the United States Government, thus, the Government has certain rights in the invention.

### FIELD OF THE INVENTION

The general field of the present disclosure are novel approaches to producing a cell-based therapy wherein certain secreted proteins and extracellular components are isolated from adipose-derived stem cells. Specifically, the present disclosure provides novel therapeutics to target and treat neurodegenerative diseases such as amyotrophic lateral sclerosis.

### BACKGROUND

Amyotrophic lateral sclerosis (ALS) is a devastating progressive neurodegenerative disease that causes the death of upper and lower motor neurons (MNs) in the central nervous system (CNS). The disease afflicts most people in prime periods of productivity in life, and it is estimated approximately 200,000 individuals in the United States live with ALS and any given time. Though a significant percentage of individuals with ALS have a genetic or hereditary form of the disease, the majority are sporadic cases with unknown etiologies. Regardless of cause, onset and progression of the disease are similar in all ALS patients, with minor initial outward symptoms followed by rapid deterioration of motor function leading to widespread paralysis, respiratory dysfunction and death. Despite the distressing and debilitating nature of ALS, no cures and limited potential treatment options exist. Therefore, identifying targets for effective therapy leading to delayed disease progression, increased quality of life, and extended lifespan are critical areas of investigation. In addition, identifying biomarkers of disease progression is incredibly important as diagnosis often occurs once the disease is in late stages and lifespan is only an average of 3-5 years after diagnosis. As the cellular and physiological processes known to influence or be involved in ALS are numerous, the complexity of the disease is a major detriment in developing effective therapies. Aside from the ubiquitous death of MNs, inflammatory and immunologic response in the spinal cord, brain and target muscles, and signal pathway changes that precede or are induced by MN death have been identified at multiple stages of disease progression. As with most neural disorders and diseases, a multifactorial approach to therapy is likely the most appropriate.

### Adipose-Derived Stem Cells

Adipose-derived stem cells (ASCs) have been shown to have utility for treating neural injury and disease. ASCs are multipotent mesenchymal stem cells that are easily obtained from adipose tissue. As such, isolating and purifying ASCs for autologous transplantation or treatment is much more accessible than for bone marrow-derived stem cells or other mesenchymal stem cells. Transplantation of these cells into neural and other tissue for studying their therapeutic effects has yielded variable responses of ASCs to the local microenvironment that affect differentiation and cell fate. See for example Tsuli et al., “Adipose-derived stem cells: Implications in tissue regeneration,” (2014) World J Stem Cells 2014, 6(3): 312-321.

In neural therapy, recent studies have shown benefits of CNS transplantation of ASCs in stroke and spinal cord injury animal models. When transplanted into the spinal cord, ASCs have demonstrated the potential to differentiate toward a neuronal phenotype and promote remodeling of the microenvironment of the injured cord including promoting serotonergic axonal regeneration. See Kolar et al., “The therapeutic effects of human adipose-derived stem cells in a rat cervical spinal cord injury model,” (2014), Stem Cells Dev 23:1659-1674. In a stroke model, transplanted ASCs reduced cell death, infarct volume, and reduced inflammatory cytokine expression in injured brain tissue when combined with mild hypothermia. See Zhao et al., “Combination of mild therapeutic hypothermia and adipose-derived stem cells for ischemic brain injury,” (2018), Neural Regen Res 13:1759-1770.

Because the microenvironment affects the function and fate of ASCs following transplantation, their beneficial properties arise from the paracrine effects of trophic and neurotrophic factor secretion by ASCs. This has promoted exploration of the use of conditioned medium from cultured ASCs as a potentially less invasive, yet still effective treatment paradigm.

The inventors have previously demonstrated the protective effects of adipose-derived stem cell conditioned medium (ASC-CM) in a mouse model of ALS (i.e. the mutant superoxide dismutase 1 (mSOD1^{G93A}) transgenic ALS mouse model). See Walker et al., “Adipose-derived stem cell conditioned medium impacts asymptomatic peripheral neuromuscular denervation in the mutant superoxide dismutase (G93A) transgenic mouse model of amyotrophic lateral sclerosis,” (2018) Restor Neurol Neurosci. 36(5): 621-627; Walker, “Adipose-derived stem cell conditioned medium for the treatment of amyotrophic lateral sclerosis: pre-clinical evidence and potential for clinical application,” (2019) Neural Regen Res 14(9):1522-1524. doi:10.4103/1673-5374.253514.

However, those studies utilized human subcutaneous adipose tissue samples that were obtained and processed, i.e. digested and separated, and then placed in endothelial growth medium consisting of in part, endothelial basal medium-2; fetal bovine serum (FBS), and the supplemental growth factors vascular endothelial growth factor (VEGF), basic fibroblast growth factor (bFGF), epidermal growth factor (EGF), and insulin-like growth factor (IGF-1). This growth medium has previously been shown to favor the growth and characteristics of vascular cells with angiogenic properties. While the secretome from such adipose-derived cells demonstrated some utility in a mouse model of ALS (see Walker et al., 2018 and Walker 2019), the inventors determined that a more specific secretome was needed in order to reverse or halt the neurodegenerative effects and improve the lifespan in a mouse model of ALS.

The present invention addresses these needs.

### SUMMARY OF THE DISCLOSURE

The inventors have discovered a novel formulation and methods for obtaining an improved adipose-derived stem cell conditioned medium (ASC-CM; i.e. secretome) and growth media with a specific profile of secreted factors capable of use in the treatment of ALS and other neurodegenerative disorders. The ASC-CM growth media is cell-free containing the critical growth factors, cytokines and other extracellular components isolated from the adipose-derived stem cells. A systemic and/or local therapy to slow disease progression and improve lifespan and quality of life in ALS patients. The disclosed ASC-CM therapy is cell-free and allows for the systemic or local administration therapy derived from large volume cultures of ASCs. The inventors disclose in the current invention, a novel formulation derived from the ASC-CM composed of particular growth factors and other components forming a reproducible specific profile of secreted factors to allow consistent treatment of ALS and other neurodegenerative disorders.

In embodiments of the current invention are provided methods of isolating and growing ASCs. In other embodiments the ASCs are grown on a large industrial scale to allow for the productions of ASC-CM growth media.

In still other embodiments, the invention provides methods for producing ASC-CM growth media containing one or more growth factors. In certain embodiments the growth factors are selected from: HGF, PDGF-AA, EGF, HB-EGF, GDNF, NT-4, bFGF, VEGF-A, TGF-B3, SCF, TCF-B2, VEGF-D, IGF-1R, M-CSF, IGF-2, NT-3, PDGF-AB, IGF-1, PLGF, bNGF, PDGF-BB, TGF-a, and GCSF. In some embodiments, the ASC-CM growth media contains all of the growth factors listed.

In yet other embodiments, the invention provides methods for producing ASC-CM growth media containing one or more cytokines. In certain embodiments, the cytokines are selected from: Pentraxin-3, PAI-1, Thromnospondin-1, MCP-1, IL-8, GROa/CXCL1, Dkk-1, Chitinase-3-like-1, MCP-3, IGFBP-3, HGF, ENA-78/CXCL5, uPAR, Osteopontin, Emmprin/CD147, MMP-9, MIC-1/GDF-15, VEGF, TFF3, IL-6 SDF-1a, Endoglin/CD105, Adiponectin, IL-17A, SHGB, IFN-gamma, Andiopoietin-2, Angiogenin, GM-CSF, IL-1a, Lipocalin-2, Vitamin D-BP, Kallikrein-3, CD40L, FGF-19 and CD30. In some embodiments, the ASC-CM growth media contains all of cytokines listed.

In certain embodiments, the invention provides compositions comprising the secretome or conditioned media, which refers to the media in which the ASCs were grown. In some embodiments, the compositions comprising the secretome include additional factors or components exogenously added.

In some embodiments, the current invention provides a method of treating ALS or other neurodegenerative diseases comprising administering a pharmaceutical composition which includes the secretome or conditioned media, i.e. ASC-CM, from the growth and culture of adipose-derived stem cells.

In other embodiments, the pharmaceutical compositions contain one or more additional growth factors or other components. Those additional other components include one or more pharmaceutically acceptable excipients.

In some embodiments, the method of treating ALS or other neurodegenerative diseases comprises administering a pharmaceutical composition which includes the secretome or condition media. i.e. ASC-CM, from the growth and culture of adipose-derived stem cells resulting in the alleviation of the symptoms of ALS. In certain embodiments, the alleviation of symptoms includes one or more of reducing muscle atrophy, halting spinal motor neuron atrophy, increasing lifespan, improving coordination, reducing muscle weakness, improving speech difficulties, and improving cognitive impairments such as learning and memory.

In still other embodiments, the current invention provides a method of treating, reducing or halting the symptoms or reversing the symptoms of neurodegenerative diseases or disorders where the neurodegenerative disease or disorder includes one or more of: Alzheimer's disease and other memory disorders, dementias, Lewy body disease, prion disease, ataxia such as Friedreich's ataxia, Huntington's disease, Parkinson's disease and Parkinson-related disorders, motor neuron disease, spinal muscle atrophy, progressive supranuclear palsy, any neurological disorder caused by a virus or other etiological agent, Tay-Sachs disease, Neiman-Pick disease, and a neuronal ceroid lipofuscinosis.

In any embodiments of the invention, the pharmaceutical composition can be administered by any pharmaceutically acceptable means. In any of the embodiments of the invention, the pharmaceutical compositions are administered one or more times per day.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. 1 is a schematic flowchart of the ASC culturing and condition protocol.

FIG. 2 is a schematic flowchart depicting the manufacturing and testing points for establishing a master adipose cell bank.

FIG. 3 a schematic flowchart depicting the manufacturing and testing points for establishing a working adipose cell bank.

FIG. 4 shows the effects of growth media on ASC proliferation. The top bar shows the cell count in a growth medium of DMEM/F12 and 10% FBS. The bottom bar shows the cell count following growth of ASC in DMEM/F12, 5% FBS and the growth factor formula of the current invention.

FIG. 5 depicts the composition of the panel of growth factors (as a percentage of positive control) for optimal ASC proliferation and production of the ASC-CM.

FIG. 6 depicts the cytokine profile (as a percentage of positive control) for optimal ASC proliferation and production of the ASC-CM.

FIG. 7A-C depicts the treatment results of mSOD1^{G93A} mice with the systemic administration (IP) of 200 μl ASC-CM or vehicle medium daily from post-natal day 70-90. Behavior was assessed at symptom onset until human endpoint: * p<0.05; FIG. 7A: lifespan; FIG. 7B: limb extension; FIG. 7C: grip endurance. The results show the improvement in all parameters following treatment with ASC-CM.

FIG. 8A-D depicts effects on the concentration of pro-inflammatory cytokines following treatment of mSOD1^{G93A} mice with the systemic administration (IP) of 200 μl ASC-CM or vehicle medium (n=4-7 per group) daily from post-natal day 70-90. * p<0.05; FIG. 8A: IFN-γ; FIG. 8B: IL-2; FIG. 8C: IL-1β1; FIG. 8D: SDF-1. The results show a significant decrease in pro-inflammatory cytokines following treating with ASC-CM.

FIG. 9 depicts the results on wet muscle weight following treatment of wild-type (WT) or mSOD1^{G93A} mice with the systemic administration (IP) of 200 μl ASC-CM or vehicle medium (n=4-7 per group) daily from post-natal day 70-90. * p<0.05; ** p<0.01; *** p<0.001; The results show an improvement in wet muscle mass in mice treated with ASC-CM.

### DETAILED DESCRIPTION

The present disclosure is based on the inventor's discovery of a novel formulation and methods for obtaining an improved adipose-derived stem cell conditioned medium (ASC-CM) and growth media with a specific profile of secreted factors capable of use in the treatment of ALS and other neurodegenerative disorders. The ASC-CM growth media is cell-free containing the critical growth factors, cytokines and other extracellular components isolated from the adipose-derived stem cells. A systemic and/or local therapy to slow disease progression and improve lifespan and quality of life in ALS patients. The disclosed ASC-CM therapy is a cell-free and allows for the systemic or local administration therapy derived from large volume cultures of ASCs.

Various quantities, such as amounts, sizes, dimensions, proportions, and the like, are presented in a range format throughout this disclosure. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is to describe particular embodiments only and is not intended to be limiting of any embodiment. As used herein, the singular forms “a,” “an”, and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless expressly stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Stem Cells.

Stem cells are promising potential treatments for multiple conditions, and researchers have demonstrated these effects broadly. Of these, adipose-derived stem cells (ASCs) are unique and have many benefits for the treatment of neural injury and disease. ASCs are multipotent mesenchymal stem cells that are easily obtained from adipose tissue. As such, isolating and purifying ASCs for autologous transplantation or treatment is much more accessible than for bone marrow-derived stem cells or other mesenchymal stem cells. Location and method of isolation influence the differentiation potential and other phenotypic characteristics of ASCs, but most are readily obtained from subcutaneous fat via lipoaspiration. In general, isolated ASCs tend to express similar surface markers to pericytes, favor differentiation toward vascular pericyte lineage, and evidence suggests these cells may be isolated from perivascular regions within the adipose tissue (Traktuev et al., 2008). Despite this seemingly vascular-centric nature of ASCs, transplantation of these cells into neural tissue for studying their therapeutic effects has yielded variable responses of ASCs to the local microenvironment that affect differentiation and cell fate.

In neural therapy, recent studies have shown benefits of CNS transplantation of ASCs in stroke and spinal cord injury animal models. When transplanted into the spinal cord, ASCs have demonstrated the potential to differentiate toward a neuronal phenotype and promote remodeling of the microenvironment of the injured cord including promoting serotonergic axonal regeneration (Kolar et al., 2014). In a stroke model, transplanted ASCs reduced cell death, infarct volume, and reduced inflammatory cytokine expression in injured brain tissue when combined with mild hypothermia (Zhao et al., 2018). Though the microenvironment affects the function and fate of ASCs following transplantation, their beneficial properties arise from the paracrine effects of trophic and neurotrophic factor secretion by ASCs. This has promoted exploration of the use of conditioned medium from cultured ASCs as a potentially less invasive, yet still effective treatment paradigm. The current invention addresses this need.

ASC-CM has been shown to be effective in the experimental treatment of a myriad of neurologic pathologies, injuries, and conditions. Recent studies have demonstrated neuroprotective and regenerative effects of ASC-CM in animal models of Parkinson's disease, hypoxic-ischemic brain injury, and peripheral nerve injury.

The inventors have previously shown that administration of human ASC-CM maintains neuromuscular innervation when given prior to onset of this early pathology (Walker et al., 2018), and late-stage treatment (at symptom onset) has proven effective at reducing MN death, delaying symptom progression, and extending lifespan in the classic mutant superoxide dismutase 1 (mSOD1)^{G93A} transgenic mouse model of ALS (Fontanilla et al., 2015).

### Stem Cell Growth and Culturing

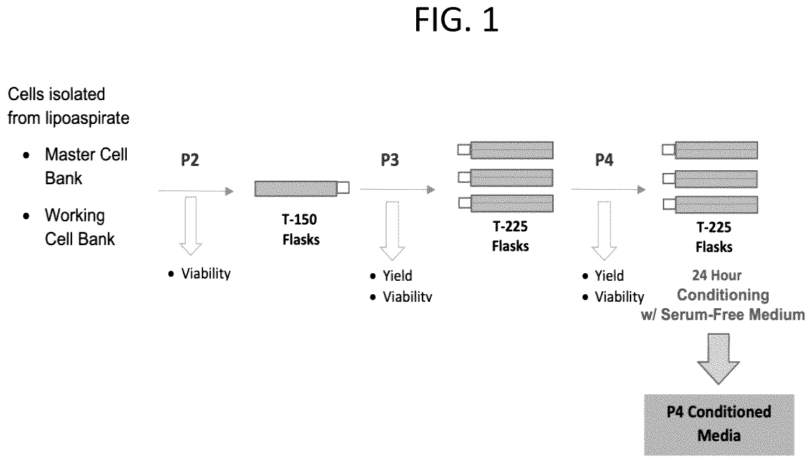

FIG. 1 is a schematic flowchart of the ASC culturing and condition protocol. The ASC and MSC Growth Formula comprises DMED/F12, 5% FBS, EGF (5 ng/mL), and ascorbic acid (250 μM).

### Adipose Stem Cell Bank

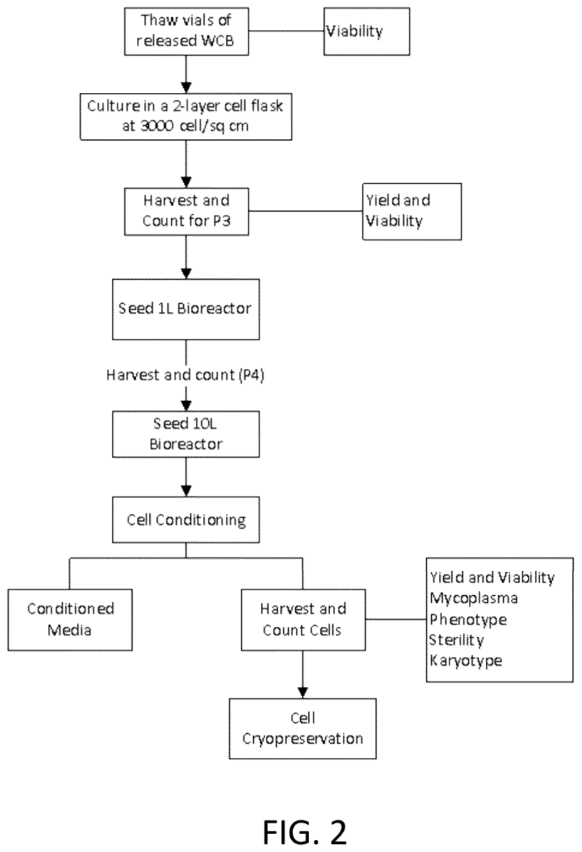

FIG. 2 is a schematic flowchart depicting the manufacturing and testing points for establishing a master adipose cell bank.

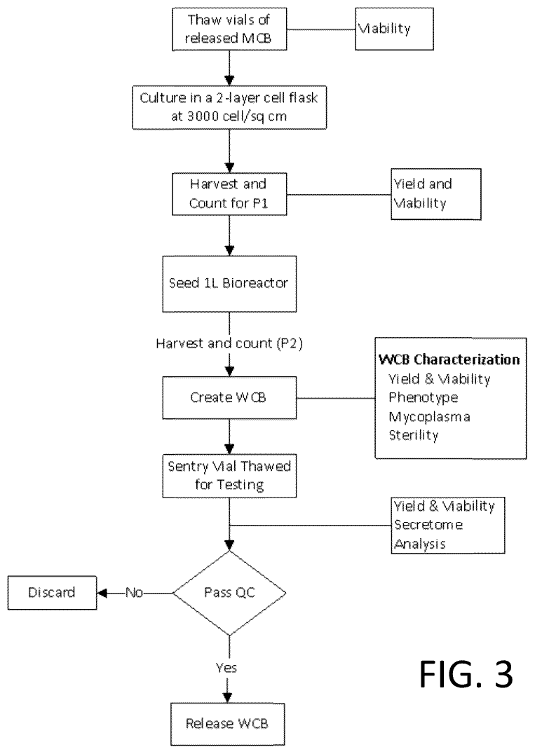

FIG. 3 a schematic flowchart depicting the manufacturing and testing points for establishing a working adipose cell bank.

### Optimal Stem Cell Growth Factor Media

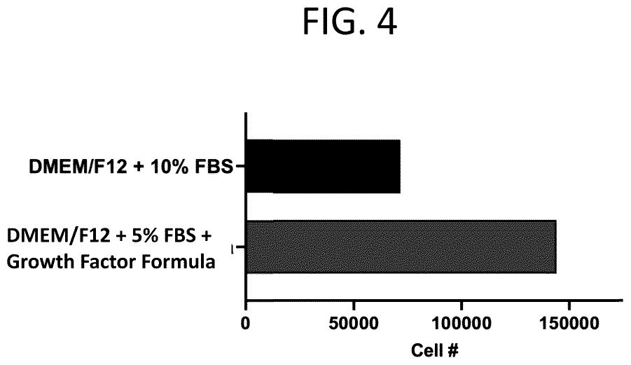

FIG. 4 shows the effects of growth media on ASC proliferation. The top bar shows the cell count in a growth medium of DMEM/F12 and 10% FBS. The bottom bar shows the cell count following growth of ASC in DMEM/F12, 5% FBS and the growth factor formula of the current invention. The optimal media can include: DMEM/F12; 5% FBS; EGF (5 ng/ml); hFGF-B (10 ng/ml); Ascorbic acid (250 uM).

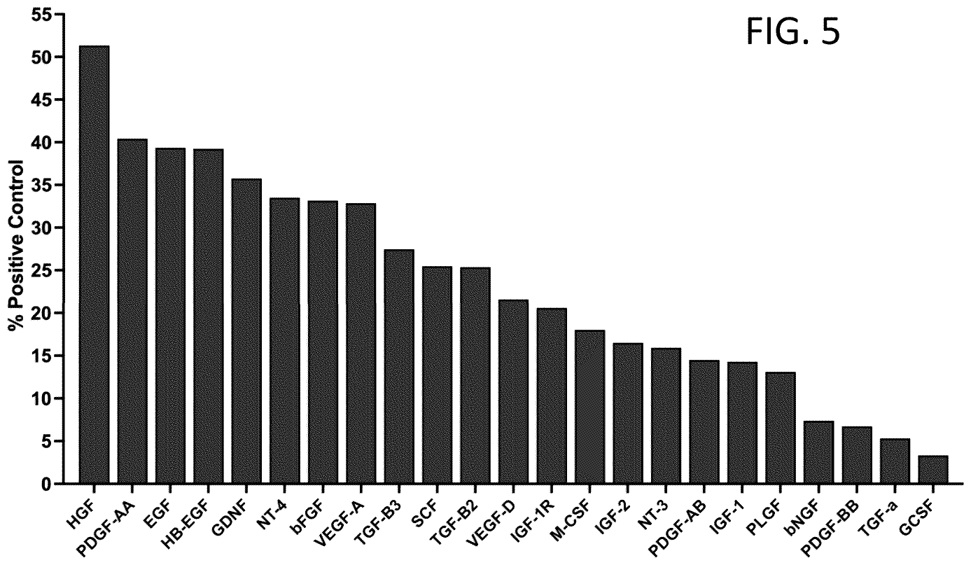

FIG. 5 depicts the composition of the panel of growth factors (as a percentage of positive control) for optimal ASC proliferation and production of the ASC-CM.

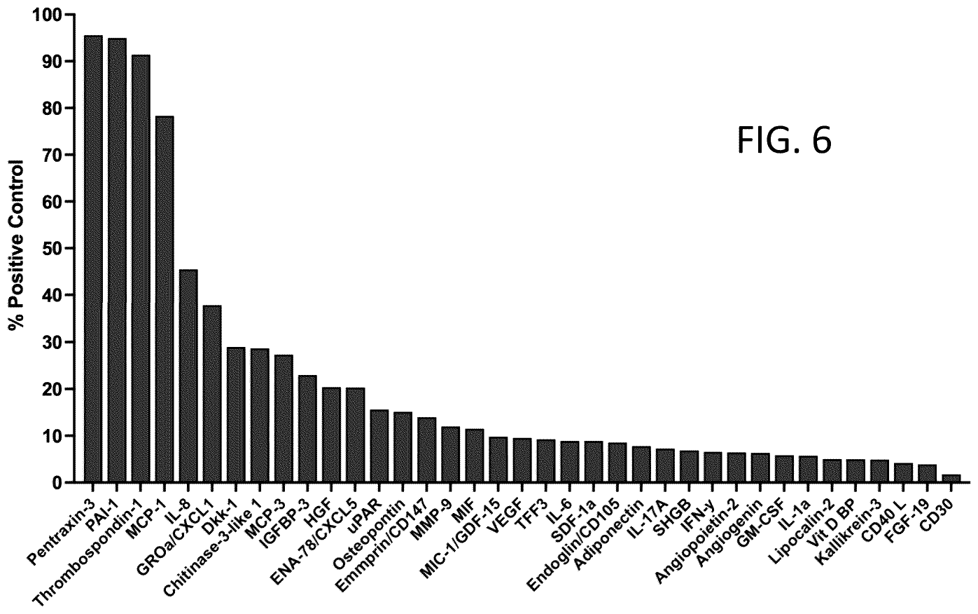

FIG. 6 depicts the cytokine profile (as a percentage of positive control) for optimal ASC proliferation and production of the ASC-CM.

### ASC-CM Secretome

The current invention provides for use of the ASC-CM secretome for use in the treatment or prevention of ALS or other neurodegenerative disorders.

It has been demonstrated that ASC-CM is a cocktail of protective and growth-inducing proteins. Hundreds of proteins are secreted by ASCs, and the benefits observed from ASC-CM therapy in neurological diseases have been attributed or confirmed to be due in large part to the various trophic factors released from the cells into the medium. Neuroprotective effects of ASC-CM are known to be highly influenced by the presence of brain-derived neurotrophic factor (BDNF) (Wei et al., 2009), nerve growth factor (Fontanilla et al., 2015), insulin-like growth factor-1 (IGF-1) (Wei et al., 2009), and glial cell line-derived neuro-trophic factor (GDNF) (Palomares et al., 2018). However, systemic and intrathecal administration of individual growth factors has shown limited or conflicting therapeutic efficacy in ALS patients.

A promising therapeutic aspect of ASC-CM, however, is the ability to deliver several beneficial neurotrophic factors in one systemic treatment approach. In showing neuroprotective, symptomatic and survival benefits in a mouse model of ALS, it is possible that many different individual factors exhibited benefits, with nerve growth factor a noted contributor to these effects (Fontanilla et al., 2015).

The inventors have demonstrated a clear influence of ASC-CM on the preservation of innervated neuromuscular junctions when administered before and through a period of initial neuromuscular disconnection in the mSOD1^{G93A} mouse model of ALS (Walker et al., 2018). In the current invention, the inventors demonstrated a cocktail of trophic factors could have played a role in imparting this benefit.

### Administration of the Secretome

In certain embodiments, secretome is administered by any means known to a person of skill in the art. The secretome may be formulated into a pharmaceutical composition. The pharmaceutical composition may be administered intraperitoneally, intravenously, subcutaneously, or orally to a patient in need. Solid dosage forms for oral administration include, as illustrative but non-limiting examples, capsules, tablets, pills, powders, thin films and granules. In solid dosage forms, the active compound may be mixed with at least one inert, pharmaceutically acceptable excipient or carrier, as described in more detail below.

In other embodiments, the secretome pharmaceutical compositions may be administered in an inhalation dosage form. In some embodiments, the pharmaceutical composition may be administered to a patient as a nasal drop (intranasally) or using a nebulization technique. A nebulizer may be used to change a liquid solution of a pharmaceutical composition into a fine mist that a patient may inhale. Using either a nasal drop or nebulization technique allows the pharmaceutical composition to travel from the olfactory bulb directly to the brain.

In some embodiments, the nebulized pharmaceutical composition may be inhaled through one or both of the mouth or the nasal passage. Without being bound to any theory, it is believed that nasal administration of the composition can take advantage of “nose-to-brain” (N2B) transport systems. Several possibilities exist for bypassing the blood-brain barrier for direct delivery to the brain. These include the draining of drugs absorbed in the nasal mucosa into the sinus and eventually to the carotid artery, where a “counter-current transfer” from venous blood to the brain may occur. Lymphatic drainage into the perivascular space from the olfactory trigeminal nerves between the central nervous system (CNS) has also been postulated as the mechanism of N2B transport.

Nebulizers are known in the art and the invention of the present disclosure can be used in connection with any nebulizer. For example, the pharmaceutical composition disclosed herein may be nebulized with an inhaler or a Buxco® Inhalation Tower All-In-One Controller.

In other embodiments, the pharmaceutical composition may be administered as in a transdermal patch or in a topical dosage form.

### Excipients

Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

### “Effective or Therapeutic Amount”

Effective or therapeutic amounts of the compositions of this disclosure include any amount sufficient to inhibit (e.g., slow or stop) the progression of ALS and/or a neurodegenerative disorder. In some embodiments, effective amounts of the compositions include any amount sufficient to inhibit (e.g., slow or stop) the deterioration of the muscular function of a patient.

The amount of secretome as the active ingredient that may be combined with the optional carrier materials to produce a single dosage form may vary depending upon the host treated and the particular mode of administration. The specific dose level for any particular patient may depend upon a variety of factors, including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

Further reference is made to the following experimental examples.

### EXAMPLES

The following examples are provided for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

Effects of Intraperitoneal Administration of ASC-CM in mSOD1^{G93A} mice

A mouse model of ALS, mSOD1^{G93A} mice, was used to demonstrate the effectiveness of ASC-CM treatment Female mSOD1^{G93A} ALS mice [B6SJL-Tg(SOD1*^{G93A})1Gur/J; Jackson Laboratories] were utilized. Female wild-type (WT) mice served as controls. For time course assessment of NMJ innervation, mSOD1^{G3A} mice were euthanized with an overdose of ketamine/xylazine at post-natal days 35, 39, 43 and 47 (n=3-4/timepoint). WT mice were euthanized at post-natal day 47 (n=3). Once anesthetized, as determined by lack of response to toe pinch stimulus, transcardial perfusion was performed with 50 ml phosphate-buffered saline (PBS, pH 7.4) followed by 4% paraformaldehyde in 0.1 M PBS for tissue fixation. Once perfused, hindlimb gastrocnemius muscles were dissected and post-fixed for 40 min, then transferred to 30% sucrose solution for cryoprotection and cryosectioning.

Human subcutaneous adipose tissue samples were obtained from lipoaspiration/liposuction procedures and prepared as previously described (Gu et al., 2013; Traktuev et al., 2008; Wang et al., 2014). In brief, human adipose tissue samples were digested in collagenase type I, filtered with 100 μm and 70 μm filters, and centrifuged at 300 g for 5 min to separate stromal cells from adipocytes. The ASC pellet was treated with red blood cell lysis buffer for 5 min at 37° C., then centrifuged at 300 g for 5 min. The supernatant was discarded, and the cell pellet was resuspended in the ASC-CM growth media of the current invention, namely, DMEM/F12, 5% FBS, EGF (5 ng/ml), hFGF-B (10 ng/ml), ascorbic acid (250 uM).

ASCs were cultured as previously described. See Walker et al. 2018. For treatment, mSOD1^{G93A} mice were randomly assigned to ASC-CM or vehicle treatment groups, and daily i.p. injections of 200 μl ASC-CM or vehicle were administered. Injections were started post-natal day 70 and continued until day 90. During treatment, the general health and overall behavior of the mice was regularly monitored by the investigators, animal facility veterinary staff and qualified veterinarians. Daily ASC-CM injections did not cause any observable outward physical or behavior changes in the mice or weight changes compared to vehicle-treated mice during the course of the study.

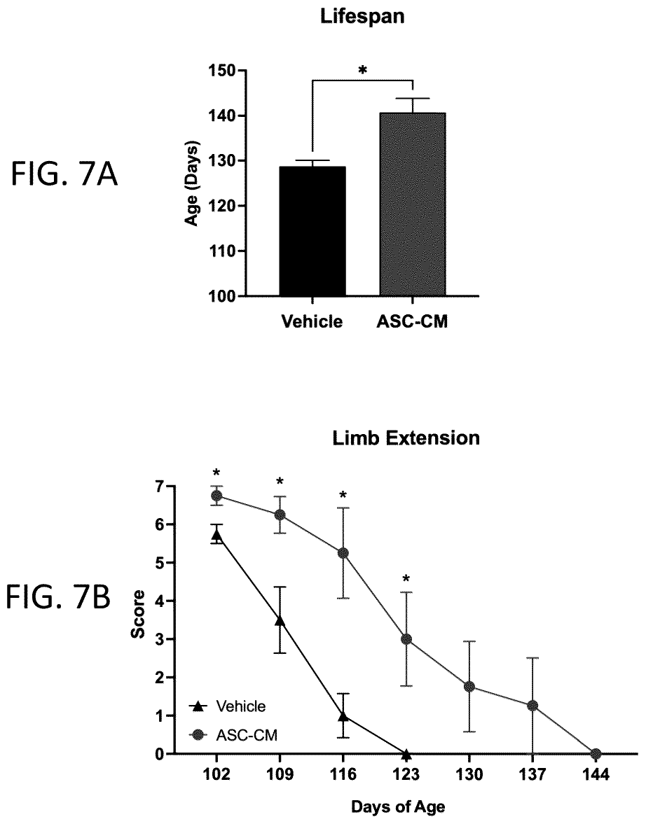

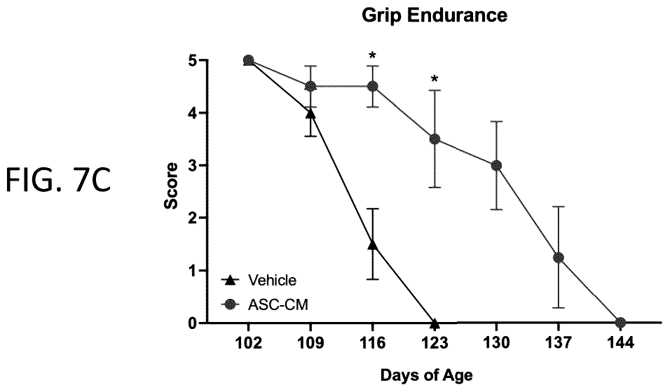

FIG. 7A-C depicts the results of treatment of mSOD1^{G93A} mice with the systemic administration (IP) of 200 μl ASC-CM or vehicle medium daily from post-natal day 70-90. Behavior was assessed at symptom onset until human endpoint: * p<0.05; FIG. 7A: lifespan; FIG. 7B: limb extension; FIG. 7C: grip endurance. The results show the improvement in all parameters following treatment with ASC-CM.

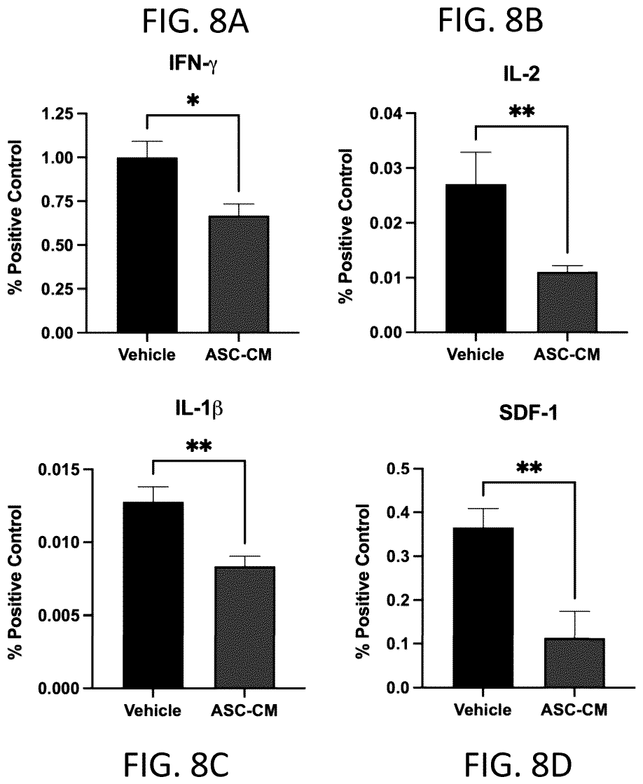

FIG. 8A-D depicts the results on the concentration of pro-inflammatory cytokines following treatment of mSOD1^{G93A} mice with the systemic administration (IP) of 200 μl ASC-CM or vehicle medium (n=4-7 per group) daily from post-natal day 70-90. * p<0.05; FIG. 8A: IFN-γ;

FIG. 8B: IL-2; FIG. 8C: IL-1β1; FIG. 8D: SDF-1. The results show a significant decrease in pro-inflammatory cytokines following treating with ASC-CM.

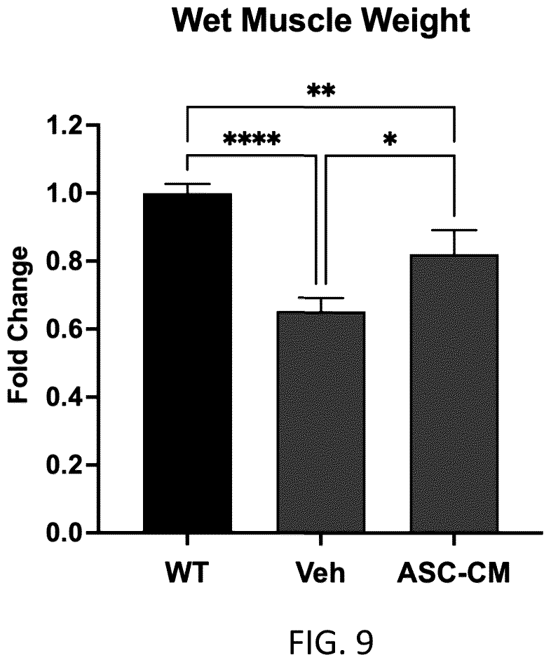

FIG. 9 depicts the results on wet muscle weight following treatment of wild-type (WT) or mSOD1^{G93A} mice with the systemic administration (IP) of 200 μl ASC-CM or vehicle medium (n=4-7 per group) daily from post-natal day 70-90. * p<0.05; ** p<0.01; *** p<0.001; The results show an improvement in wet muscle mass in mice treated with ASC-CM.

As will be appreciated from the descriptions herein, a wide variety of aspects and embodiments are contemplated by the present disclosure, examples of which include, without limitation, the aspects and embodiments listed below:

A novel formulation and methods for obtaining an improved adipose-derived stem cell conditioned medium (ASC-CM) and growth media with a specific profile of secreted factors capable of use in the treatment of ALS and other neurodegenerative disorders. The ASC-CM growth media is cell-free containing the critical growth factors, cytokines and other extracellular components isolated from the adipose-derived stem cells. A systemic and/or local therapy to slow disease progression and improve lifespan and quality of life in ALS patients. The disclosed ASC-CM therapy is cell-free and allows for the systemic or local administration therapy derived from large volume cultures of ASCs.

Methods of isolating and growing ASCs. In other embodiments, the ASCs are grown on a large industrial scale to allow for the productions of ASC-CM growth media.

Methods for producing ASC-CM growth media containing one or more growth factors. In certain embodiments the growth factors are selected from: HGF, PDGF-AA, EGF, HB-EGF, GDNF, NT-4, bFGF, VEGF-A, TGF-B3, SCF, TGF-B2, VEGF-D, IGF-1R, M-CSF, IGF-2, NT-3, PDGF-AB, IGF-1, PLGF, bNGF, PDGF-BB, TGF-a, and GCSF.

Methods for producing ASC-CM growth media containing one or more cytokines. In certain embodiments, the cytokines are selected from: Pentraxin-3, PAI-1, Thromnospondin-1, MCP-1, IL-8, GROa/CXCL1, Dkk-1, Chitinase-3-like-1, MCP-3, IGFBP-3, HGF, ENA-78/CXCL5, uPAR, Osteopontin, Emmprin/CD147, MMP-9, MIC-1/GDF-15, VEGF, TFF3, IL-6 SDF-1a, Endoglin/CD105, Adiponectin, IL-17A, SHGB, IFN-gamma, Andiopoietin-2, Angiogenin, GM-CSF, IL-1a, Lipcalin-2, Vitamin D-BP, Kallikrein-3, CD40L, FGF-19 and CD30.

Compositions comprising the secretome or conditioned media refer to the media in which the ASCs were grown. In some embodiments, the compositions comprising the secretome include additional factors or components exogenously added.

Methods of treating ALS or other neurogenerative diseases comprising a administering pharmaceutical composition that includes the secretome or condition media, i.e. ASC-CM, from the growth and culture of adipose-derives stem cells. The pharmaceutical compositions contain one or more additional growth factors or other components. Those additional other components include one or more pharmaceutically acceptable excipients.

Method of treating ALS or other neurodegenerative diseases comprising a administering a pharmaceutical composition which includes the secretome or condition media, i.e. ASC-CM, from the growth and culture of adipose-derives stem cells resulting in the alleviation of the symptoms of ALS. In certain embodiments, the alleviation of symptoms includes one or more of reducing muscle atrophy, halting spinal motor neuron atrophy, increasing lifespan, improving coordination, reducing muscle weakness, improving speech difficulties, and improving cognitive impairments such as learning and memory.

Methods of treating, reducing or halting the symptoms or reversing the symptoms of neurodegenerative diseases or disorders where the neurodegenerative disease or disorder includes one or more of: Alzheimer's disease and other memory disorders, dementias, Lewy body disease, prion disease, ataxia such as Friedreich's ataxia, Huntington's disease, Parkinson's disease and Parkinson-related disorders, motor neuron disease, spinal muscle atrophy, progressive supranuclear palsy, any neurological disorder caused by a virus or other etiological agent, Tay-Sach's disease, Neiman-Pick disease, and a neuronal ceroid lipofuscinosis.

Methods wherein the pharmaceutical composition can be administered by any pharmaceutically acceptable means and wherein the pharmaceutical compositions are administered one or more times per day.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of obtaining an improved adipose-derived stem cell conditioned medium (ASC-CM) or secretome with a profile of secreted factors capable of use in the treatment of amyotrophic lateral sclerosis (ALS) and other neurodegenerative disorders comprising isolating, growing and stimulating adipose-derived stem cella in a growth media, wherein the growth media comprises DMEM/F12; 5% FBS; EGF (5 ng/ml); hFGF-B (10 ng/ml); and ascorbic acid (250 uM) to produce the adipose-derived stem cell conditioned medium (ASC-CM) or secretome.

2. The method of claim 1, wherein the ASC-CM comprises one or more growth factors selected from: HGF, PDGF-AA, EGF, HB-EGF, GDNF, NT-4, bFGF, VEGF-A, TGF-B3, SCF, TGF-B2, VEGF-D, IGF-1R, M-CSF, IGF-2, NT-3, PDGF-AB, IGF-1, PLGF, bNGF, PDGF-BB, TGF-a, and GCSF.

3. The method of claim 1, wherein the ASC-CM comprises one or more cytokines selected from: Pentraxin-3, PAI-1, Thromnospondin-1, MCP-1, IL-8, GROa/CXCL1, Dkk-1, Chitinase-3-like-1, MCP-3, IGFBP-3, HGF, ENA-78/CXCL5, uPAR, Osteopontin, Emmprin/CD147, MMP-9, MIC-1/GDF-15, VEGF, TFF3, IL-6 SDF-1a, Endoglin/CD105, Adiponectin, IL-17A, SHGB, IFN-gamma, Andiopoietin-2, Angiogenin, GM-CSF, IL-1a, Lipcalin-2, Vitamin D-BP, Kallikrein-3, CD40L, FGF-19 and CD30.

4. A pharmaceutical composition comprising the ASC-CM produced from the method of claim 1, wherein the compositions further include one or more additional factors or components exogenously added.

5. A method of treating amyotrophic lateral sclerosis (ALS) comprising administration of the pharmaceutical composition of adipose-derived stem cell conditioned medium (ASC-CM) or secretome of claim 4.

6. The method of claim 5, wherein the administration of the pharmaceutical composition results in the alleviation of the symptoms of ALS.

7. The method of claim 6, wherein the alleviation of symptoms includes one or more of reducing muscle atrophy, halting spinal motor neuron atrophy, increasing lifespan, improving coordination, reducing muscle weakness, improving speech difficulties, and improving cognitive impairments such as learning and memory.

8. A method of treating, reducing, or halting the symptoms or reversing the symptoms of a neurodegenerative disorder comprising administration of a pharmaceutical composition of claim 4.

9. The method of claim 8, wherein the neurodegenerative disease or disorder includes one or more of: Alzheimer's disease and other memory disorders, dementias, Lewy body disease, prion disease, ataxia such as Friedreich's ataxia, Huntington's disease, Parkinson's disease and Parkinson-related disorders, motor neuron disease, spinal muscle atrophy, progressive supranuclear palsy, any neurological disorder caused by a virus or other etiological agent, Tay-Sachs disease, Neiman-Pick disease, and a neuronal ceroid lipofuscinosis.

10. The pharmaceutical composition of claim 4, wherein the pharmaceutical composition can be administered by any pharmaceutically acceptable mode of administration.

11. The pharmaceutical composition of claim 10, wherein the pharmaceutical composition is administered one or more times per day.

12. The method for obtaining an improved adipose-derived stem cell conditioned medium (ASC-CM) of claim 1, comprising:
i) isolating subcutaneous adipose tissue samples were obtained from a patient, wherein the tissue sample is obtained from lipo-aspiration or liposuction;
ii) digesting the adipose tissue samples in filtered collagenase type I;
iii) separating the adipose-derived stem cells from the adipose tissue adipocytes via centrifugation to obtain a first pellet containing the adipose-derived stem cells;
iv) treating the adipose-derived stem cell pellet with a red blood cell lysis buffer and further centrifuging to obtain a second adipose-derived stem cell pellet; and,
v) resuspending the second adipose-derived stem cell pellet in a growth medium to stimulate the adipose-derived stem cells to obtain the adipose-derived stem cell conditioned medium (ASC-CM), wherein the growth medium comprises DMEM/F12; 5% FBS; EGF (5 ng/ml); hFGF-B (10 ng/ml); and ascorbic acid (250 uM) to produce the adipose-derived stem cell conditioned medium (ASC-CM) or secretome.

13. The method of claim 12, wherein the ASC-CM comprises one or more growth factors selected from the group consisting of HGF, PDGF-AA, HB-EGF, GDNF, NT-4, bFGF, VEGF-A, TGF-B3, SCF, TGF-B2, VEGF-D, IGF-1R, M-CSF, IGF-2, NT-3, PDGF-AB, IGF-1, PLGF, bNGF, PDGF-BB, TGF-a, and GCSF.

14. (canceled)

15. The method of claim 12, wherein the ASC-CM further contains one or more cytokines.

16. The method of claim 15, wherein the one or more cytokines are selected from the group consisting of Pentraxin-3, PAI-1, Thromnospondin-1, MCP-1, IL-8, GROa/CXCL1, Dkk-1, Chitinase-3-like-1, MCP-3, IGFBP-3, HGF, ENA-78/CXCL5, uPAR, Osteopontin, Emmprin/CD147, MMP-9, MIC-1/GDF-15, VEGF, TFF3, IL-6 SDF-1a, Endoglin/CD105, Adiponectin, IL-17A, SHGB, IFN-gamma, Andiopoietin-2, Angiogenin, GM-CSF, IL-1a, Lipcalin-2, Vitamin D-BP, Kallikrein-3, CD40L, FGF-19 and CD30.

17.-18. (canceled)
